(19) United States
(12) Patent Application Publication
(10) Pub. No.: US 2022/0323167 A1
(43) Pub. Date: Oct. 13, 2022

(54) SURGICAL SYSTEM

(71) Applicant: MEDICAROID CORPORATION, Kobe-shi (JP)

(72) Inventor: Kaoru TAKAHASHI, Kobe-shi (JP)

(73) Assignee: MEDICAROID CORPORATION, Kobe-shi (JP)

(21) Appl. No.: 17/714,783

(22) Filed: Apr. 6, 2022

(30) Foreign Application Priority Data
Apr. 7, 2021 (JP) 2021-065114
Jul. 19, 2021 (JP) 2021-118459

(51) Int. Cl. A61B 34/30

(52) U.S. Cl. CPC A61B 34/30; A61B 2034/305; A61B 2017/00398

(57) ABSTRACT

A surgical system according to one or more embodiment may be configured such that, upon receiving a command to rotate first and second rotation members by predetermined rotation angles for closing first and second jaw members to each other, a controller is configured, when a path length of a first elongate element between the first jaw member and the first rotation member changes, to control a first actuator to rotate the first rotation member by a first rotation angle corrected according to change in the path length of the first elongate element, and when a path length of a second elongate element between the second jaw member and the second rotation member changes, to control a second actuator to rotate the second rotation member by a second rotation angle corrected according to change in the path length of the second elongate element.

### CROSS REFERENCE TO RELATED APPLICATIONS

This application claims priority based on 35 USC 119 from prior Japanese Patent Applications No. 2021-118459 filed on Jul. 19, 2021 and No. 2021-065114 filed on Apr. 7, 2021, the entire contents of all of which are incorporated herein by reference.

### BACKGROUND

The disclosure may relate to a surgical system.

In a related art, there has been known a robotic surgical system including a robot arm, a tool (a surgical instrument) connected to a distal end of the robot arm, and an input handle (an operation part). For example, Patent Document 1 (U.S. Pat. No. 6,594,552) discloses a robotic surgical system configured to perform torque control in movements of a surgical instrument based on user operation received by an operation part. Specifically, the robotic surgical system is configured to operate the surgical instrument with four degrees of freedom by four motors in a surgical instrument mounting portion provided at a robot arm. The surgical instrument includes a shaft, two jaw members that are provided on a distal side of the shaft and configured to be opened and closed with each other, and four rotation members (four driven members) provided on a proximal side of the shaft and configured to be respectively driven by the four motors in the surgical instrument mounting portion. Two of the rotation members are connected to the two jaw members via cables. The robotic surgical system performs torque control to operate the jaw members by rotating the rotation members until the torque applied to the motor reaches a predetermined torque. A patient-side cart of the robotic surgical system is equipped with three such robot arms.

Patent Document 1: U.S. Pat. No. 6,594,552

### SUMMARY

It may be preferable to reduce the size of the patient-side cart of the robotic surgical system, in particular, to reduce the size of the robot arm around the surgical field so as to secure the work area of an assistant doctor or the like during the operation. In order to reduce the size of the robot arm around the surgical field, it may be effective to reduce the size of the four motors arranged in the surgical instrument mounting portion. In order to reduce the size of the motor, it may be effective to use a motor whose size is smaller than a conventional motor and a speed reducer having a reduction ratio higher than that of a conventional speed reducer to operate the surgical instrument by using the output of the small motor. However, in a case of the robot surgical system described in Patent Document 1 performing torque control of the motor that operates the surgical instrument, if a speed reducer having a reduction ratio higher than that of a conventional speed reducer is used, a change in a torque or a current value of the motor is less likely to be reflected to the behavior of members provided on a distal end side of the surgical instrument, and thus it may be difficult to detect the behavior of the members provided on the distal end side of the surgical instrument by monitoring the change in the torque or the current value of the motor.

In order to solve this problem, it may be effective to control a rotation angle of the motor (a rotation angle of the rotation member of the surgical instrument) to operate the surgical instrument, instead of controlling the torque of the motor that drives the surgical instrument. In a case where the two jaw members, which are members of the end effector of the surgical instrument, are scissors, a shearing force is generated between the two jaw members by further rotating the motors by predetermined rotation angles after the two jaw members are closed. In a case where the two jaw members, which are members of the end effector of the surgical instrument, are graspers, a gripping force is generated between the two jaw members by further rotating the motors by predetermined rotation angles after the two jaw members are closed.

However, in a configuration in which the surgical instrument is operated by such control, for example, when a path length(s) of the cable(s) between the rotation member(s) and the jaw member(s) changes, the shearing force changes in the case of the scissors, and the gripping force changes in the case of the graspers.

An object of an embodiment of the disclosure may be to provide a surgical system capable of reducing changes in shearing force, gripping force, or the like between jaw members even when a path length of a cable between a rotation member and a jaw member changes.

An aspect of the disclosure may be a robotic surgical system that may include: a patient-side apparatus that includes a robot arm including, at a distal end side of the robot arm, an attachment portion to which a surgical instrument is attached; an operator-side apparatus including an operation part configured to receive an operation for operating the surgical instrument; and a controller configured to control an operation of the surgical instrument based on an operation from the operation part. The surgical instrument includes: a shaft; a first support body connected to a distal end portion of the shaft; a second support body supported at a distal end portion of the first support body and configured to be rotatable about a first axis orthogonal to an axial direction of the shaft with respect to the first support body; a first jaw member including a first pulley portion supported at a distal end portion of the second support body and configured to be rotatable about a second axis orthogonal to the axial direction of the shaft and the first axis with respect to the second support body; a second jaw member including a second pulley portion supported at the distal end portion of the second support body and configured to be rotatable about the second axis with respect to the second support body; a first elongate element for operating the end effector; a second elongate element for operating the end effector; a first rotation member provided on a proximal end side of the shaft and configured to be rotated to drive the first elongate element; and a second rotation member provided on the proximal end side of the shaft and configured to be rotated to drive the second elongate element. The first jaw member and the second jaw member are configured to be closed to each other by the first pulley portion being rotated in a first direction and the second pulley portion being rotated in a second direction opposite to the first direction. The attachment portion of the robot arm includes a first actuator configured to drive the first rotation member and a second actuator configured to drive the second rotation member of the surgical instrument attached to the attachment portion. Upon receiving from the operation part an operation to rotate the first and second rotation members by first and second predetermined rotation angles respectively for closing the first and second jaw members to each other, the controller is configured, when a path length of the first elongate element between the first jaw member and the first rotation member changes, to control the first actuator to rotate the first rotation member by a first rotation angle corrected according to the change in the path length of the first elongate element, and when the path length of the second elongate element between the second jaw member and the second rotation member changes, to control the second actuator to rotate the second rotation member by a second rotation angle corrected according to the change in the path length of the second elongate element.

According to the aspect described above, the controller monitors a condition in which the path length of the first elongate element between the first jaw member and the first rotation member changes, and controls, upon receiving from the operation part the operation to rotate the first rotation member by the predetermined rotation angle, the first actuator to rotate the first rotation member by a rotation angle corrected according to the condition. Accordingly, when the path length of the first elongate element is changed, the first actuator is controlled to rotate the first rotation member by the corrected rotation angle, so that change in the shearing force or the gripping force between the jaw members can be reduced.

According to one or more embodiments of the disclosure, even when the operation of the surgical instrument is controlled based on the rotation angle of the motor (or the rotation angle of the rotation member of the surgical instrument), it may be possible to suppress a decrease in the shearing force and the gripping force of the surgical instrument.

### BRIEF DESCRIPTION OF DRAWINGS

FIG. 1 is a diagram illustrating a view of a configuration of a surgical system according to an embodiment;

FIG. 2 is a diagram illustrating a view of a configuration of a patient-side apparatus according to an embodiment;

FIG. 3 is a diagram illustrating a view of a configuration of a robot arm of the patient-side apparatus according to an embodiment;

FIG. 4 is a diagram illustrating a perspective view of a state of an embodiment where a surgical instrument is attached to the robot arm through an adaptor;

FIG. 5 is a diagram illustrating an exploded perspective view of a state of an embodiment where the surgical instrument is attached to the robot arm through the adaptor;

FIG. 6 is a diagram illustrating a perspective view of the surgical instrument according to an embodiment as seen from below;

FIG. 7 is a diagram illustrating a perspective view of a state where a cover part is detached from a base body of the surgical instrument according to an embodiment;

FIG. 8 is a diagram illustrating a perspective view of the adaptor according to an embodiment as seen from below;

FIG. 9 is a diagram illustrating a view of an actuator according to an embodiment;

FIG. 10 is a diagram illustrating a plan view of a state where the cover part is detached from the base body of the surgical instrument according to an embodiment;

FIG. 11 is a diagram illustrating a perspective view of an end effector of the surgical instrument according to an embodiment;

FIG. 12 is a diagram illustrating a perspective view of the end effector of the surgical instrument according to an embodiment;

FIG. 13 is a diagram illustrating a perspective view of the end effector of the surgical instrument according to an embodiment;

FIG. 14 is a block diagram illustrating a view of a configuration of a controller of the surgical system according to an embodiment;

FIG. 15 is a diagram illustrating a perspective view of the end effector of the surgical instrument with a wrist portion being bent according to an embodiment;

FIG. 16 is a diagram illustrating a relationship between a bending angle and a slack of a wire;

FIG. 17 is a diagram illustrating a perspective view of the end effector of the surgical instrument with the wrist portion being bent according to an embodiment;

FIG. 18 is a diagram illustrating a flowchart for explaining a control method of the surgical system according to an embodiment; and

FIG. 19 is a diagram illustrating a perspective view of a grip type end effector according to an embodiment.

### DETAILED DESCRIPTION

Descriptions are provided hereinbelow for one or more embodiments of the disclosure based on the drawings. In the respective drawings referenced herein, the same constituents are designated by the same reference numerals and duplicate explanation concerning the same constituents is omitted. All of the drawings are provided to illustrate the respective examples only.

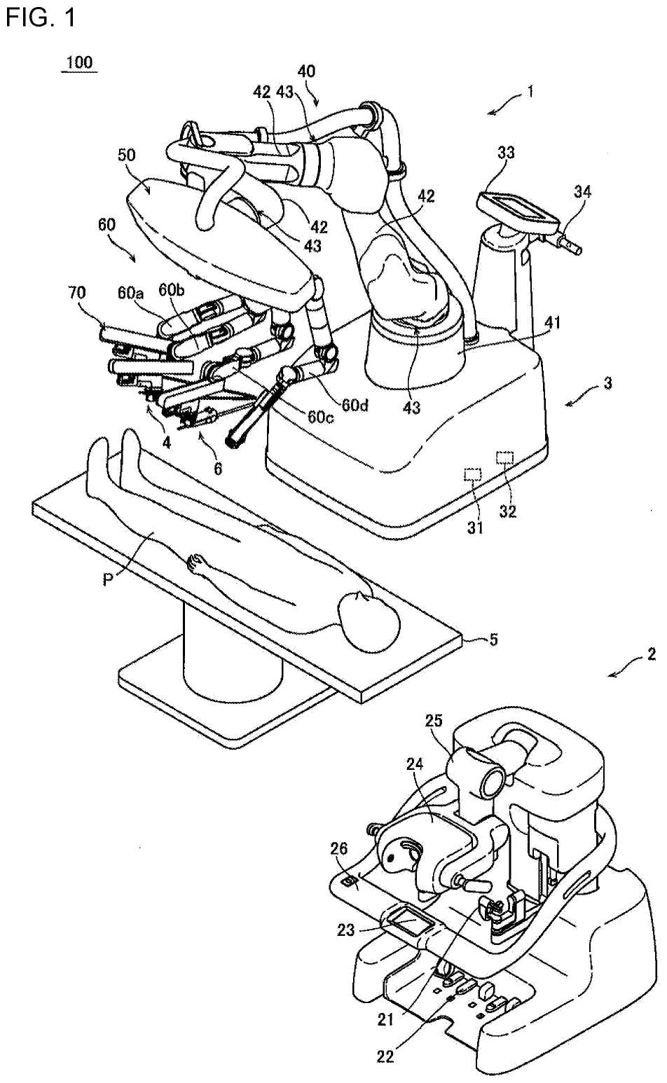

A configuration of a surgical system 100 according to one or more embodiments is described with reference to FIGS. 1 to 18. The surgical system 100 includes a medical manipulator 1 serving as a patient-side apparatus and a remote control apparatus 2 serving as an operator-side apparatus to operate the medical manipulator 1. The medical manipulator 1 is provided with a medical trolley 3 and is thus configured to be movable. The remote control apparatus 2 is provided at a location away from the medical manipulator 1. The medical manipulator 1 is configured to be remotely operated by the remote control apparatus 2. An operator or a surgeon inputs to the remote control apparatus 2 an instruction that causes the medical manipulator 1 to perform a desired operation. The remote control apparatus 2 transmits the input instruction to the medical manipulator 1. The medical manipulator 1 operates in response to the received instruction. The medical manipulator 1 is disposed in a surgery room, as a sterile field, which is sterilized.

The remote control apparatus 2 is disposed inside the surgery room or outside the surgery room, for example. The remote control apparatus 2 includes operation manipulator arms 21, operation pedals 22, a touch panel 23, a monitor 24, a support arm 25, and a support bar 26. The operation manipulator arms 21 constitute operation handles for the operator to input the instruction. Specifically, the operation manipulator arms 21 receive an amount of movement input by the operator O to operate surgical instruments 4. The monitor 24 is a display device of a scope type configured to display an image captured by an endoscope. The support arm 25 supports the monitor 24 in such a manner that the height of the monitor 24 is adjusted to the height of the face of the operator. The touch panel 23 is disposed on the support bar 26. When a sensor(s) (not illustrated) provided in the vicinity of the monitor 24 detects the head of the operator, the medical manipulator 1 can be operated by the operator using the remote control apparatus 2. The operator operates the operation manipulator arms 21 and the operation pedals 22, while viewing the surgical site displayed on the monitor 24. With this, the instruction is input to the remote control apparatus 2. The instruction that is input to the remote control apparatus 2 is transmitted to the medical manipulator 1. Note that the operation manipulator arm 21 is an example of an “operation part.”

The medical trolley 3 is provided with a control unit 31 that controls the operation of the medical manipulator 1 and a storage 32 that stores therein programs for controlling the operation of the medical manipulator 1. Based on the instruction inputted to the remote control apparatus 2, the control unit 31 of the medical trolley 3 controls the operation of the medical manipulator 1. Note that the control unit 31 is an example of a “control device” or a “controller.”

Further, the medical trolley 3 is provided with an input device 33. The input device 33 is configured to accept operations to move or change posture of a positioner 40, an arm base 50, and robot arms 60 (hereinafter may be referred to as arms 60), mainly to prepare for surgery before the surgery.

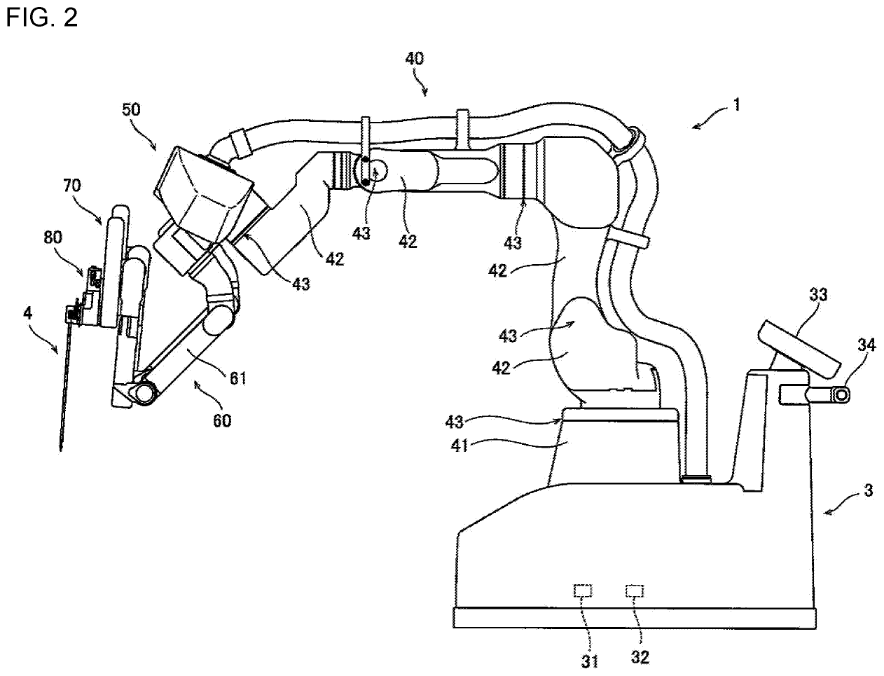

The medical manipulator 1 illustrated in FIGS. 1 and 2 is disposed in the surgery room. The medical manipulator 1 includes the medical trolley 3, the positioner 40, the arm base 50, and the arms 60. The arm base 50 is attached to a distal end of the positioner 40. The arm base 50 is a relatively long rod shape (elongate shape). Base portions (proximal end portions) of the arms 60 are attached to the arm base 50. Each of the arms 60 is configured such that the arm 60 is able to take a folded posture (storage posture). The arm base 50 and the arms 60 are used with being covered with a sterile drape.

The positioner 40 is configured as a 7-axis articulated robot. The positioner 40 is disposed on the medical trolley 3. The positioner 40 is configured to move the arm base 50. Specifically, the positioner 40 is configured to move the position of the arm base 50 three-dimensionally.

The positioner 40 includes a base portion 41 and link portions 42 connected to the base portion 41. The link portions 42 are connected to each other via joints 43.

As illustrated in FIG. 1, to the distal end of each of the arms 60, the surgical instrument 4 is attached. The surgical instruments 4 include, for example, an instrument, an endoscope 6, and the like that are replaceable.

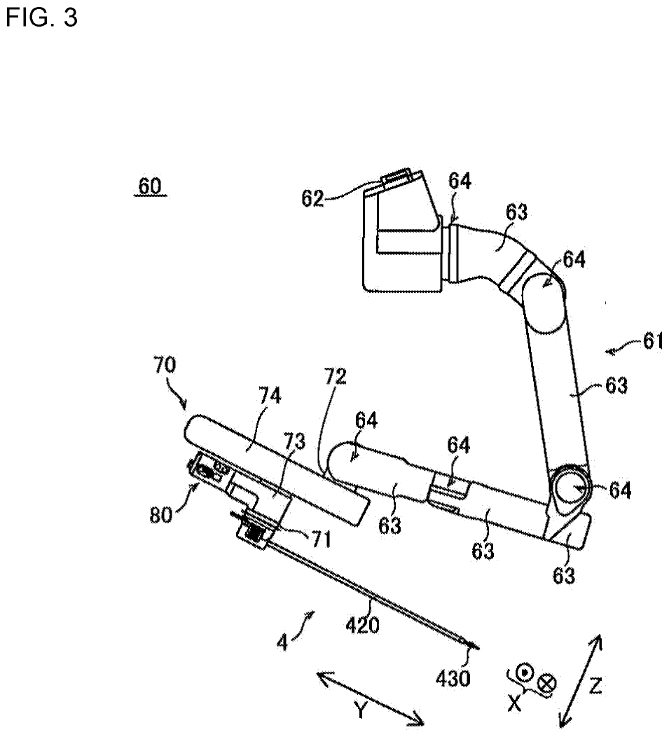

As illustrated in FIG. 3, forceps as an end effector 430 is provided to the distal end of the surgical instrument. Note that the forceps include two end effector members (jaws) 430a and 430b. The end effector members 430a and 430b are examples of a “first jaw member” and a “second jaw member”, respectively.

Next, a configuration of the arm 60 is described in detail.

As illustrated in FIG. 3, the arm 60 includes an arm section 61 (the base portion 62, the link portions 63, the joint portions 64) and a translation movement mechanism 70 provided at the distal end portion of the arm section 61. The arm 60 is configured such that the distal end portion thereof is three-dimensionally movable with respect to the proximal side (the arm base 50) of the arm 60. Each arm 60 has eight degrees of freedom. The plural arms 60 have the same configuration as each other.

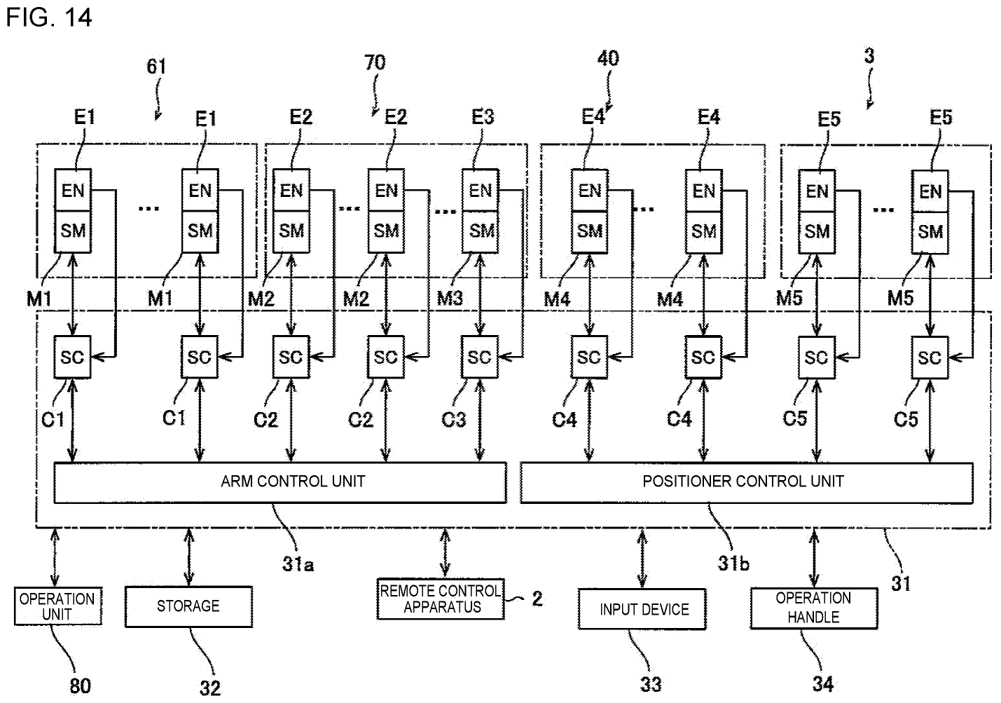

The translation movement mechanism 70 is provided on a side of the distal end of the arm section 61. The surgical instrument 4 is attached to the translation movement mechanism 70. The translation movement mechanism 70 translationally moves the surgical instrument 4 in the insertion direction of the surgical instrument 4 into a patient P. The translation movement mechanism 70 is configured to translationally move the surgical instrument 4 relative to the arm section 61. Specifically, the translation movement mechanism 70 is provided with a holder 71 configured to hold the surgical instrument 4. The holder 71 accommodates therein the servomotors M2 (see FIG. 14). The servomotors M2 are configured to rotate rotors (rotation members) provided in the surgical instrument 4. The forceps is operated by rotating the rotors in the surgical instrument 4.

The arm section 61 is configured as a 7-axis articulated robot arm. The arm section 61 includes the base portion 62 that connects the arm section 61 to the arm base 50 and the plural link portions 63 connected to the base portion 62. The plural link portions 63 are connected to each other via the joints 64.

The translation movement mechanism 70 is configured to translationally move the holder 71 along the Y direction so as to translationally move the surgical instrument 4 attached to the holder 71 along the Y direction (the extending direction or the longitudinal direction of the shaft 420). The translation movement mechanism 70 includes a proximal side link unit 72 connected to the distal end of the arm section 61, a distal side link unit 73, and a connecting link unit 74 provided between the proximal side link unit 72 and the distal side link unit 73. The holder 71 is provided at the distal side link unit 73.

The connecting link unit 74 of the translation movement mechanism 70 functions as a double speed mechanism that makes a movement speed of the distal side link unit 73 along the Z direction with respect to the proximal side link unit 72 twice as a movement speed of the proximal side link unit 72 along the Y direction with respect to the connecting link unit 74. The translation movement mechanism 70 is configured to translationally move the surgical instrument 4 attached to the holder 71 along the Y direction by moving the distal side link unit 73 with respect to the proximal side link unit 72 along the Z direction. The distal end of the arm section 61 is configured such that the proximal side link unit 72 is connected thereto in such a manner that the proximal side link unit 72 is rotatable about a rotational axis extending in the X direction orthogonal to the Y direction.

As illustrated in FIG. 1, the endoscope 6 is attached to one of the plural arms 60 (for example, the arm 60b), and the surgical instruments 4 other than the endoscope 6 are attached to the other arms 60 (for example, the arms 60a, 60c, and 60d). Specifically, for surgery, the endoscope 6 is attached to one of the four arms 60, and the surgical instruments 4 (forceps, etc.) other than the endoscope 6 are attached to the other three arms 60. In the state where the endoscope 6 is attached to the arm 60, the pivot position for the endoscope 6 is set to the arm 60 to which the endoscope 6 is attached. Further, in the state where a pivot position setting device (not illustrated) is attached to the arm 60 to which the surgical instrument 4 other than the endoscope 6 is attached, the pivot position for the surgical instrument 4 is set to the arm 60 to which the surgical instrument 4 other than the endoscope 6 is attached. The endoscope 6 is attached to one of two arms 60 (arms 60b and 60c) arranged in the central area among the four arms 60 arranged adjacent to each other. That is, the pivot position is individually set for each of the plurality of arms 60.

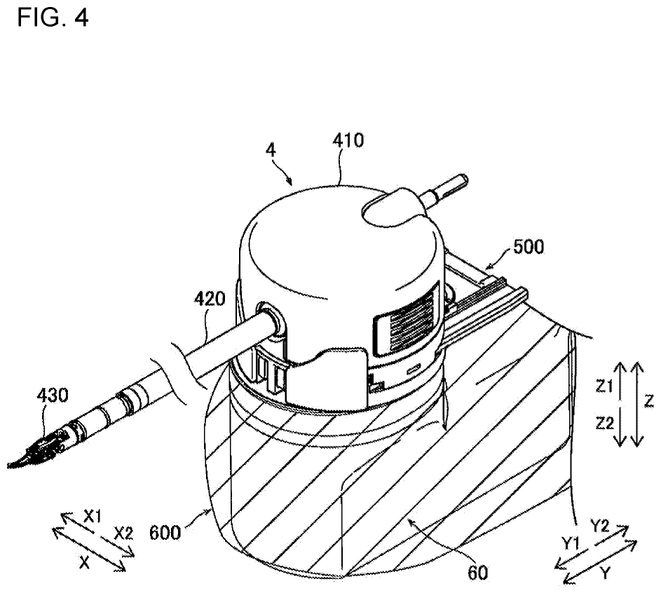

With reference to FIGS. 4 to 13, the configurations of the surgical instrument 4, an adaptor 500, and a drape 600 are described.

In this description, the direction in which the surgical instrument 4 extends (the axial direction S of a shaft 420) is referred to as the Y direction. The direction in which the surgical instrument 4 and the adaptor 500 are adjacent to each other is defined as a Z direction, the surgical instrument 4 side along the Z direction is defined as a Z1 direction, and the opposite side of the Z1 direction is defined as a Z2 direction. Further, the direction orthogonal to the Y direction and the Z direction is referred to as an X direction, one side along the X direction is referred as an X1 direction, and the other side along the X direction is referred to as an X2 direction.

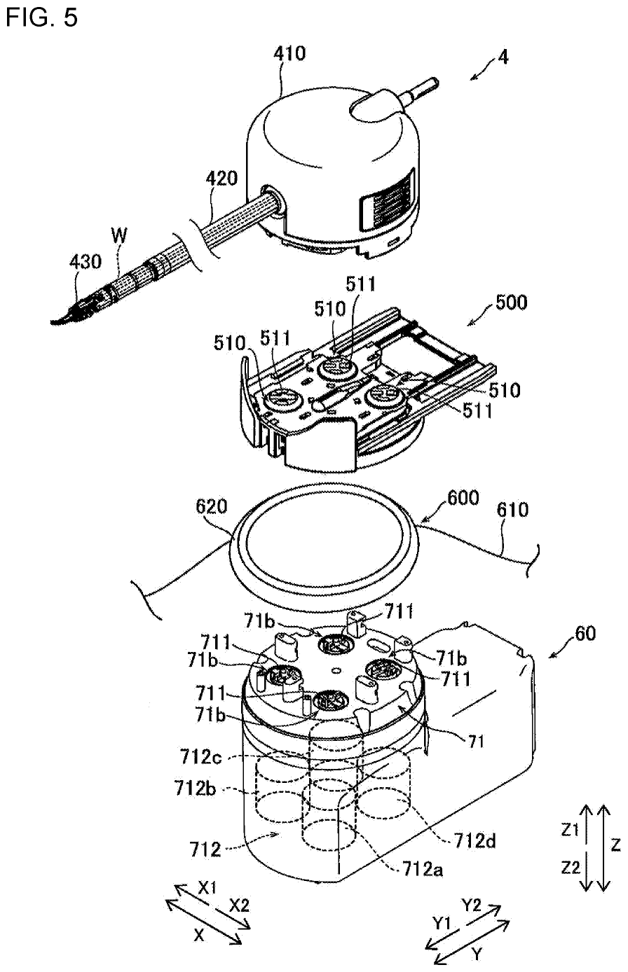

As illustrated in FIGS. 4 and 5, the surgical instrument 4 is detachably connected to a holder 71 of the arm 60 of the surgical system 10. Specifically, the surgical instrument 4 is detachably connected to the arm 60 through the adaptor 500. The adaptor 500 is a drape adaptor configured to sandwich a sterile drape 600 to cover the arm 60, between the adaptor 500 and the holder 71 of the arm 60.

The surgical instrument 4 is attached to the Z1 side of the adaptor 500. The adaptor 500 is attached to the Z1 side of the arm 60.

As illustrated in FIG. 5, the drape 600 includes a body section 610 that covers the arm 60 and an attachment section 620 sandwiched between the arm 60 and the adaptor 500. The body section 610 is made of a flexible film member. The flexible film member is made of a resin material, such as thermoplastic polyurethane and polyethylene. The body section 610 includes an opening so that the holder 71 of the arm 60 is engageable with the adaptor 500. To the opening of the body section 610, the attachment section 620 is provided. The attachment section 620 is made of a resin mold member. The resin mold member is made of a resin member such as polyethylene terephthalate. The attachment section 620 is formed harder (less flexible) than the body section 610. The attachment section 620 includes an opening so that the holder 71 is engageable with the adaptor 500.

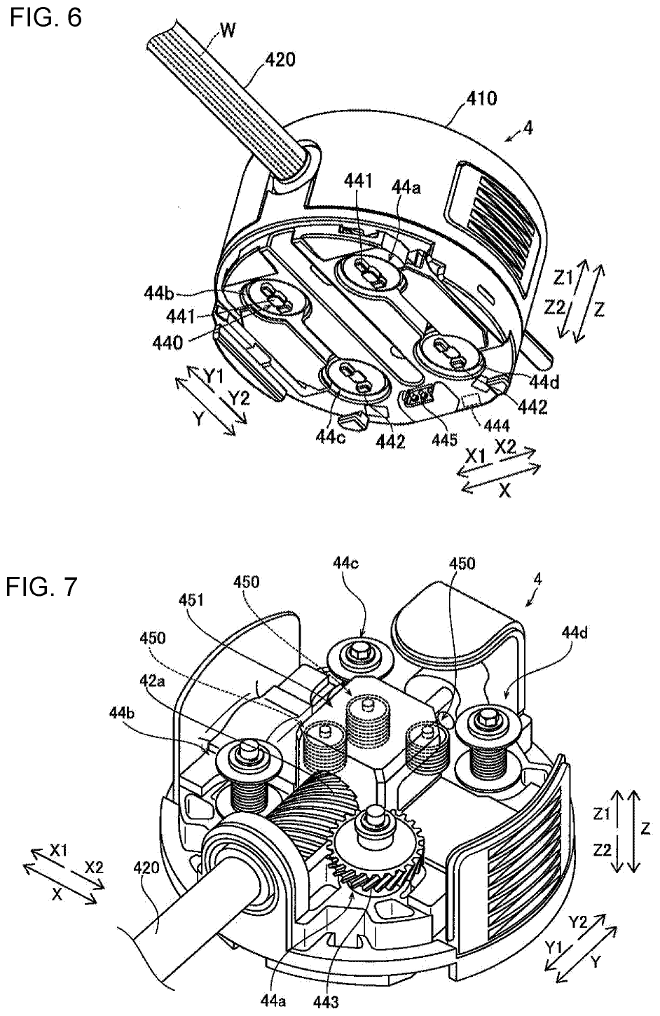

As illustrated in FIGS. 5 and 6, the surgical instrument 4 includes plural (four) rotation members 44a, 44b, 44c, and 44d. The rotation members 44a, 44b, 44c, and 44d are provided within a housing 410 and are rotatable about the respective rotation axes extending along the Z axis. The plural rotation members 44a, 44b, 44c, and 44d are provided to operate (drive) the end effector 430. The rotation members 44a, 44b, 44c, and 44d are connected to the end effector 430 via elongate elements W (wires) passing through the shaft 420. With this, rotations of the rotation members 44a, 44b, 44c, and 44d drive the elongate element W (the wire), which operate (drive) the end effector 430. In addition, the rotation member 44a is connected to the shaft 420 through gears 42a (see FIG. 7). With this, the shaft 420 is rotated by the rotation of the rotation member 44a. Note that the rotation members 44c, 44d, and 44b are examples of a “first rotation member”, a “second rotation member”, and a “third rotation member” in the disclosure, respectively.

To transmit driving forces from the holder 71 of the arm 60 to the end effector 430, the rotation members 44a, 44b, 44c, and 44d respectively include an engagement portion 440 including a projection 441 or 442, which is respectively engaged with a corresponding transmission member 510 of the adaptor 500. The projection 441 or 442 is projected from the Z2 side surface of the rotation members 44a, 44b, 44c, and 44d toward the side of the adaptor 500 (the Z2 side). The projections 441 and 442 are arranged in a straight line. The protrusions 441 provided to the rotation members 44a and 44b have different shapes from that of the protrusions 442 provided to the rotation members 44c and 44d.

As illustrated in FIG. 5, the adaptor 500 includes a plurality (four) of the drive transmission members 510. The drive transmission members 510 are configured to transmit driving forces from the arm 60 to the rotation members 44a, 44b, 44c, and 44d of the surgical instrument 4. That is, the drive transmission members 510 are provided so as to correspond to the rotation members 44a, 44b, 44c, and 44d of the surgical instrument 4, respectively. The drive transmission members 510 are rotatable about the respective rotation axes, which extend along the Z direction.

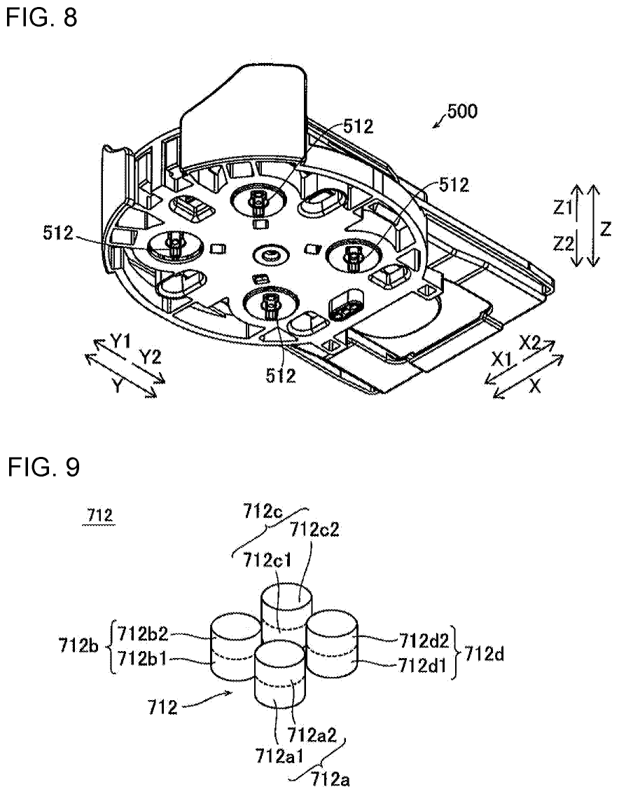

Each of the drive transmission members 510 includes an engagement portion 511 including an engagement recess which is engaged with the projection 441 or 442 of the corresponding rotation member 44a, 44b, 44c, or 44d of the surgical instrument 4. The engagement recess provided to the engagement portion 511 is located at the surgical instrument 4 side (the Z1 side) of the drive transmission member 510 and is recessed from the Z1 side surface of the drive transmission member 510, toward the Z2 direction, opposite to the surgical instrument 4 side. Each of the drive transmission members 510 is provided at the Z2 side surface thereof with the engagement portion 512 illustrated in FIG. 8 including the engagement recess, which is respectively engaged with a projection provided to an engagement portion 711 of a corresponding drive part 71b of the holder 71.

The holder 71 of the arm 60 includes plural (four) drive parts 71b. The plural drive parts 71b are provided corresponding to the plural (four) drive transmission members 510 of the adaptor 500. Each of the drive parts 71b includes the engagement portion 711 and an actuator 712. The actuators 712 include four actuators 712a, 712b, 712c, and 712d. As illustrated in FIG. 9, the actuator 712a includes a servomotor 712a1 and a speed reducer 712a2, the actuator 712b includes a servomotor 712b1 and a speed reducer 712b2, the actuator 712c includes a servomotor 712c1 and a speed reducer 712c2, and the 712d includes a servomotor 712d1 and a speed reducer 712d2. Reduction ratios of the speed reducers 712a2 to 712d2 are 10 to 150. The speed reducers 712a2 to 712d2 convert the amount of rotation (rotation angle) of the servomotors 712a1 to 712d1 to 1/10 to 1/150 times and output the reduced rotation amount (the reduced rotation angle) so as to rotate the engaging portions 711, the drive transmission members 510 engaged with the engagement portions 711, the engagement portions 440 engaged with the drive transmission members 510, and the rotation members 44a to 44d connected to the engagement portions 440. It may be preferable that the reduction ratio of the speed reducers 712a2 to 712d2 is 30 to 120, the reduction ratio of the speed reducer 712a2 is 100, and the reduction ratio of the speed reducers 712b2 to 712d2 is 50. Note that the actuators 712a, 712b, 712c, and 712d are examples of a “first actuator”, a “second actuator”, and a “third actuator.”

The engagement projection provided to the engagement portion 711 is engaged with the engagement recess provided to the engagement portion 512 of the corresponding drive transmission member 510. The engagement projection of the engagement portion 711 is projected from the Z1 side surface of the drive part 71b toward the Z1 side (the adaptor 500 side).

The actuators 712 include the servomotors 712a1 to 712d1, the speed reducers 712a2 to 712d2, and encoders (encoders E2 in FIG. 14) that detect the rotation angles of the servomotors 712a1 to 712d1. The servomotors 712a1 to 712d1 and the speed reducers 712a2 to 712d2 of the actuator 712 are configured to drive the engagement portions 711 to rotate about the rotational axes thereof extending in the Z direction. Thereby, the drive transmission members 510 of the adaptor 500 engaged with the engagement portions 711 can be rotated about the rotational axes thereof extending in the Z direction, and the rotation members 44a, 44b, 44c, and 44d of the surgical instrument 4 engaged with the drive transmission members 510 can be rotated about the rotational axes thereof.

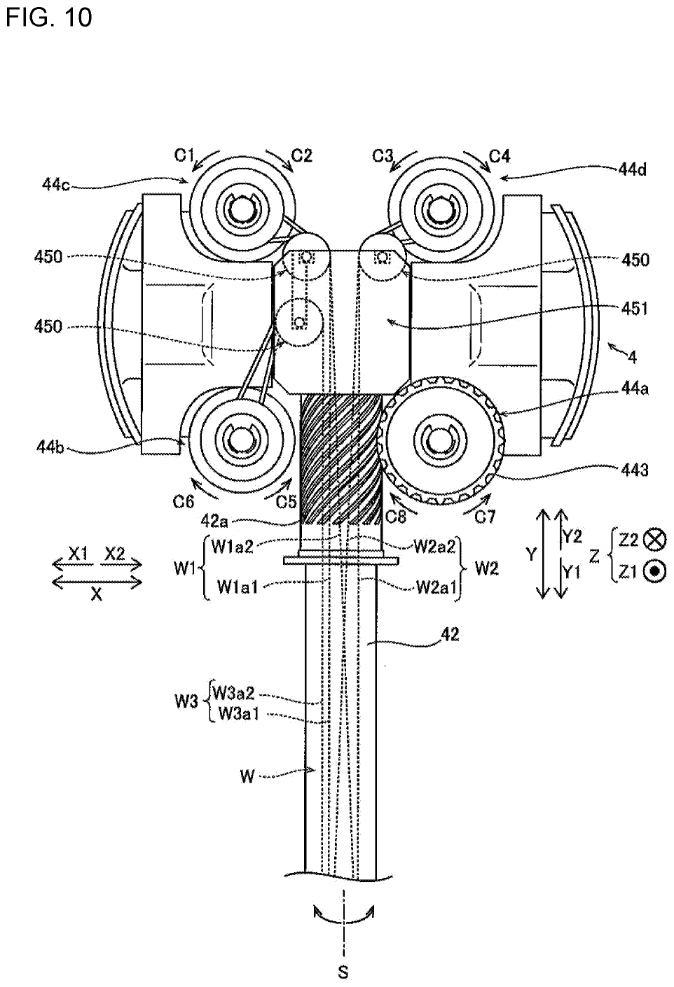

As illustrated in FIGS. 7 and 10, the elongate elements W are wound around the rotation members 44b to 44d of the surgical instrument 4. Specifically, the end portion of a first portion W3a1 of a third elongate element W3 is wound clockwise around an upper portion of the rotation member 44b, and the end portion of a second portion W3a2 of the third elongate element W3 is wound counterclockwise around a lower portion of the rotation member 44b.

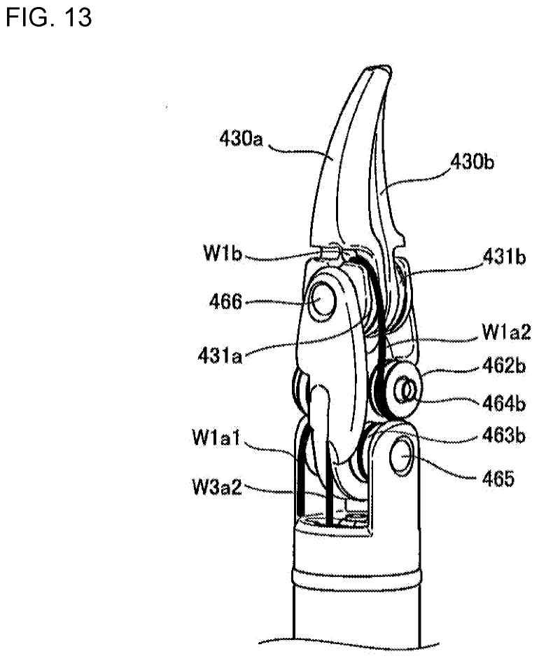

A first elongate element W1 includes a first portion W1a1, a second portion W1a2, and a stopper or an attachment W1b (see FIG. 13) provided between the first portion W1a1 and the second portion W1a2. The attachment W1b is fixed to the end effector member 430a. The end portion of the first portion W1a1 of the first elongate element W1 is wound counterclockwise around the upper portion of the rotation member 44c. The end portion of the second portion W1a2 of the first elongate element W1 is wound clockwise around the lower portion of the rotation member 44b. Note that the attachment W1b is an example of a “first attachment.”

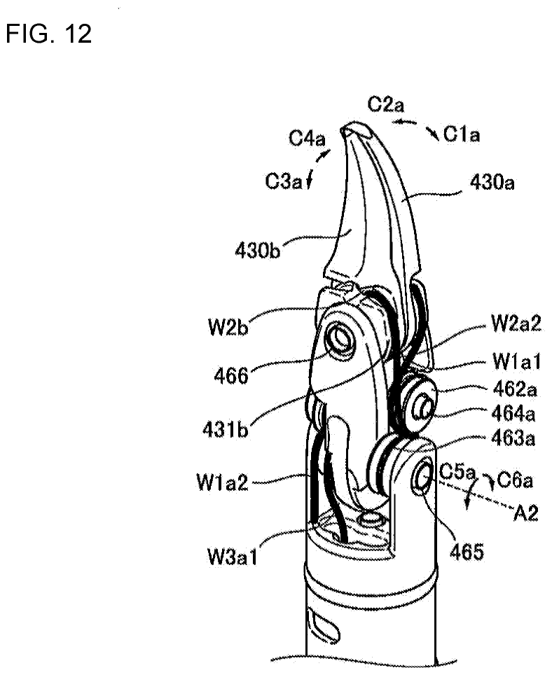

Further, a second elongate element W2 includes a first portion W2a1, a second portion W2a2, and a stopper or an attachment W2b (see FIG. 12) provided between the first portion W2a1 and the second portion W2a2. The attachment W2b is fixed to the end effector member 430b. Further, the end portion of the first portion W2a1 of the second elongate element W2 is wound counterclockwise around the upper portion of the rotation member 44d. The end portion of the second portion W2a2 of the second elongate element W2 is wound clockwise around the lower part of the rotation member 44d. Note that the attachment W2b is an example of a “second attachment.”

The elongate elements W extend from the rotation members 44b to 44d through the shaft 420 to the end effector 430, are wound around the end effector 430, and return to the rotation members 44b to 44d through the shaft 420. Further, the elongate elements W are wound around built-in pulleys 450 in the housing 410, respectively. The built-in pulleys 450 are retained in the housing 410 by a pulley retainer 451.

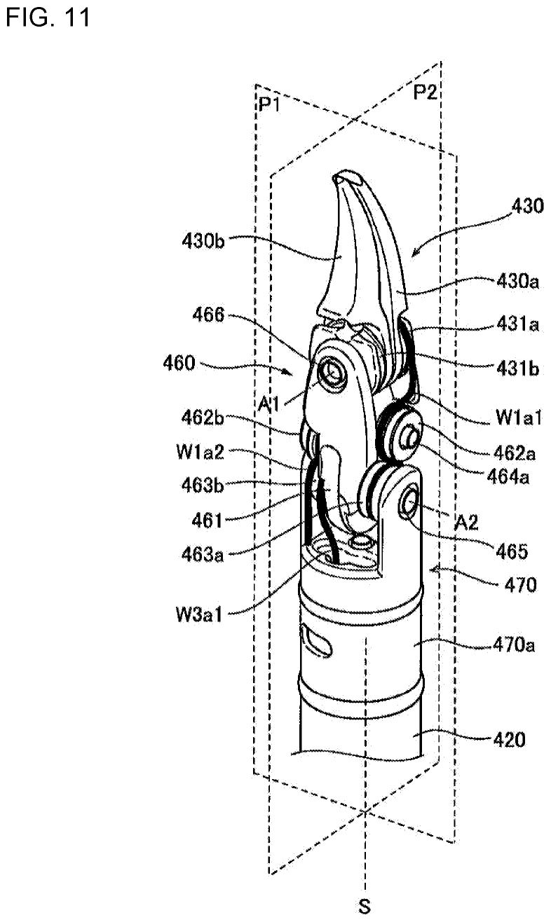

As illustrated in FIGS. 10 and 11, when the rotation member 44c is rotated about the axis thereof, the end effector member 430a of the pair of the end effector members 430a and 430b of the end effector 430 is operated. Specifically, the rotation member 44c is rotated by the servomotor 712c1 to drive the first elongate element W1, which extends through the inside of the shaft 420 and connects the end effector member 430a and the rotation member 44c. When the rotation member 44c is rotated in the C1 direction (see FIG. 10) to draw the first portion W1a1 of the first elongate element W1, the end effector member 430a is driven to move in the C1a direction (see FIG. 12) which is a direction that the end effector member 430a opens. When the rotation member 44c is rotated in the C2 direction (see FIG. 10), which is opposite to the C1 direction, to drawn the second portion W1a2 of the first elongate element W1, the end effector member 430a is driven to move in the C2a direction (see FIG. 12) which is a direction that the end effector member 430a closes. Note that in FIG. 11, the second elongate element W2 is omitted.

By being rotated about the axis thereof, the rotation member 44c operates one (430a) of the pair of the end effector members 430a and 430b of the end effector 430. Specifically, the rotation member 44d is rotated by the servomotor 712d1 to drive the second elongate element W2, which extends through the inside of the shaft 420 and connects the end effector member 430b and the rotation member 44d. When the rotation member 44b is rotated in the C3 direction (see FIG. 10) so as to draw the first portion W2a1 of the second elongate element W2, the end effector member 430b is driven to move in the C3a direction (see FIG. 12) that the end effector member 430b opens. When the rotation member 44d is rotated in the C4 direction (see FIG. 10), which is opposite to the C3 direction, to draw the second portion W2a2 of the second elongate element W2, the end effector member 430b is driven to move in the C4a direction (see FIG. 12) which is a direction that the end effector member 430b closes.

By being rotated about the rotation axis thereof, the rotation member 44b operates a distal clevis 460, which is a wrist portion of the end effector 430. Specifically, the rotation member 44b is rotated to drive the third elongate element W3. When the rotation member 44b is rotated in the C5 direction (see FIG. 10) to draw the first portion W3a1 of the third elongate element W3, the distal clevis 460 is driven to move in the C5a (see FIG. 12). When the rotation member 44b is rotated in the C6 direction (see FIG. 10), which is opposite to the C5 direction, the distal clevis 460 is driven to move in the C6a direction (see FIG. 12), which is opposite to the C5a direction.

When the rotation member 44a is rotated about the rotation axis thereof with the gear portion 443 of the rotation member 44a being engaged with the gear portion 42a connected to the proximal end of the shaft 420, the shaft 420 is rotationally driven to rotate the end effector 430. Specifically, when the rotation member 44a is rotated in the C7 direction (see FIG. 10), the shaft 420 is driven to rotate in a first direction and thus the end effector 430 is driven to rotate in the first direction. To the contrary, when the rotation member 44a is rotated in the C8 direction (see FIG. 10), the shaft 420 is driven to rotate in a second direction opposite to the first direction and thus the end effector 430 is driven to rotate in the second direction. The shaft 420 is driven to rotate by the rotation of the actuator 712a.

In other words, as illustrated in FIG. 10, the rotation member 44c is arranged on the proximal end side of the shaft 420, and is rotated by the rotation of the actuator 712c to drive the first elongate element W1. The rotation member 44d is arranged on the proximal end side of the shaft 420, and is rotated by the rotation of the actuator 712d to drive the second elongate element W2. The end effector member 430a and the end effector member 430b are arranged at the distal end of the shaft 420 via the distal clevis 460.

In the state where the rotation angle of the shaft 420 is zero, the second portion W1a2 of the first elongate element W1 that drives the end effector member 430a and the second portion W2a2 of the second elongate element W2 that drives the end effector member 430b are arranged so as to intersect each other in the shaft 420. Note that the first portion W1a1 of the first elongate element W1 that drives the end effector member 430a and the first portion W2a1 of the second elongate element W2 that drives the end effector member 430b are arranged parallel to each other along the rotation axis S of the shaft 420.

In FIG. 11, the first portion W1a1 of the first elongate element W1 is guided by inner pulleys, which are pulleys that are provided on the side closer to the second plane P2 in a group of first pulleys 462a and a group of second pulleys 463a, and is led to the rotation member 44c along the axis S of the shaft 420. To the contrary, in FIG. 13, the second portion W1a2 of the first elongate element W1 is guided by outer pulleys, which are pulleys that are provided on the side farther from the second plane P2 in a group of first pulleys 462b and a group of second pulleys 463b, and is led to the rotation member 44c across the rotation axis S of the shaft 420. Therefore, as illustrated in FIG. 10, the first portion W1a1 of the first elongate element W1 is arranged substantially parallel to the axis S of the shaft 420, and the second portion W1a2 of the first elongate element W1 is arranged so as to intersect the axis S. For the same reason as for the first elongate element W1, the first portion W2a1 of the second elongate element W2 is arranged substantially parallel to the axis S, and the second portion W2a2 of the second elongate element W2 is arranged so as to intersect the axis S. Note that the first pulleys 462a are an example of a “second pulley”, and an “eighth pulley.” The first pulleys 462b are examples of a “fourth pulley” and a “sixth pulley.” The second pulleys 463a are examples of a “first pulley” and a “seventh pulley.” The second pulleys 463b are examples of a “third pulley” and a “fifth pulley.”

Further, as illustrated in FIG. 10, the first elongate element W1 and the second elongate element W2 are arranged so as to be twisted 180 degrees with respect to the axis S of rotation of the shaft 420 in the shaft 420 between the end effector member 430a and the end effector member 430b in the shaft 420. Specifically, in a state where the distal clevis 460 to which the end effector member 430a and the end effector member 430b are attached is rotated 180 degrees around the rotation axis S of the shaft 420 after the attachment W1b of the first elongate element W1 and the attachment W2b of the second elongate element W2 are attached to the end effector member 430a and the end effector member 430b, the end portion of the first portion W1a1 and the end portion of the second portion W1a2 of the first elongate element W1 are wound around the rotation member 44c, and the end portion of the first portion W2a1 and the end portion of the second portion W2a2 of the second elongate element W2 are wound around the rotation member 44d.

As illustrated in FIGS. 11 to 13, the surgical instrument 4 is, for example, an electrosurgical instrument such as a monopolar curved scissors. The surgical instrument 4 includes the conductive end effector 430, the conductive distal clevis 460 that rotatably supports the end effector 430 about a first axis A1, and a conductive proximal clevis 470 that rotatably supports the distal clevis 460 about a second axis A2. The surgical instrument 4 includes the shaft 420 having a cylindrical shape and connected to the proximal clevis 470.

As illustrated in FIG. 11, the end effector 430 includes two end effector members 430a and 430b. Each of the end effector members 430a and 430b is, for example, a jaw member made of a conductive member such as stainless steel or the like.

Specifically, the end effector member 430a includes a pulley portion 431a and the end effector members 430b includes a pulley portion 431b. The end effector member 430a is configured to change the posture thereof along with the movement of the first elongate element W1 (the first portion W1a1 and the second portion W1a2) wound around the pulley portion 431a. More specifically, as illustrated in FIG. 13, the attachment W1b formed in a circular-column shape of the first elongate element W1 is engaged with the pulley portion 431a of the end effector member 430a. As the first elongate element W1 moves, the pulley portion 431a rotates about a first axis A1, which will be described later, and thus the end effector member 430a rotates about the first axis A1. As illustrated in FIG. 12, the end effector member 430b includes the pulley portion 431b. The end effector member 430b is configured to change the posture thereof along with the movement of the second elongate element W2 (the first portion W2a1 and the second portion W2a2) wound around the pulley portion 431b. Here, the case has been described in which the end effector 430 is a pair of scissors; however, the end effector 430 may be an end effector other than a pair of scissors, such as graspers 700 as illustrated in FIG. 19, for example. As illustrated in FIG. 19, a first end effector member 730a and a second end effector member 730b of a surgical instrument 730 constitute a gripping type end effector configured to grip an object. The pulley portion 431a and the pulley portion 431b are examples of a “first pulley portion” and a “second pulley portion”, respectively.

The distal clevis 460 includes: the pulley portion 461; the first pulley group (462a) which includes two pulleys 462a rotatable about a first shaft portion 464a; the first pulley group (462b) which includes two pulleys 462b rotatable about a first shaft portion 464b; the second pulley group (463a) and the second pulley group (463b), each of which includes two pulleys 463a and 463b rotatable about a second shaft portion 465; and a third shaft portion 466. The first pulley group 462a, the second pulley group 463a, and the first shaft portion 464a are arranged on one side of the distal clevis 460 with respect to the second plane P2 illustrated in FIG. 11. The first pulley group 462b, the second pulley group 463b, and the first shaft portion 464b are arranged on the other side of the distal clevis 460 with respect to the second plane P2 illustrated in FIG. 11. Each pulley included in the first pulley groups 462a and 462b has a smaller diameter than each pulley included in the second pulley groups 463a and 463b.

At the distal end side (the end effector 430 side) of the distal clevis 460, a pair of shaft holes are formed. The third shaft portion 466, which rotatably supports the pulley portion 431a of the end effector member 430a and the pulley portion 431b of the end effector member 430b, is inserted in the pair of shaft holes. The third shaft portion 466 is a shaft member formed in a cylindrical column shape extending along the first axis A1. The third shaft portion 466 is supported by the pair of shaft holes. The first axis A1 extends along a direction substantially orthogonal to the axis S of the shaft 420. The first axis A1 extends in the direction substantially orthogonal to the first plane P1 and substantially parallel to the second plane P2.

The pulley portion 461 is provided on a proximal end side (the shaft 420 side) of the distal clevis 460 and is rotatably supported about the second axis A2 by the proximal clevis 470. Specifically, the pulley portion 461 is rotatably supported by the second shaft portion 465 which is supported by the proximal clevis 470. The second axis A2 extends along the direction substantially orthogonal to the axis S of the shaft 420 and substantially orthogonal to a direction parallel to the first axis A1. The second axis A2 extends in the direction substantially parallel to the first plane P1 and substantially orthogonal to the second plane P2. The pulley portion 461 includes a pulley groove formed along a circumferential direction of the second axis A2. The distal clevis 460 is configured to change the posture thereof as the third elongate element W3 wound around the pulley portion 461 thereof moves. More specifically, a cylindrical-column-shaped attachment of the third elongate element W3 is engaged with the pulley portion 461. When the second elongate element W3 moves, the pulley portion 461 rotates about the second rotational axis A2 and thus rotates the distal clevis 460 about the second rotational axis A2. The cylindrical-column-shaped attachment is provided between the first portion W3a1 and the second portion W3a2 of the third elongate element W3. The third elongate element W3 is driven by the actuator 712b.

The two pulleys of the first pulley group 462a are rotatably supported by the first shaft portion 464a. Two pulleys of the second pulley group 463a are rotatably supported by the second shaft portion 465. In FIG. 11, the pulley that is provided on the side closer to the second plane P2 in the first pulley group 462a (the inner pulley of the first pulley group 462a) and the pulley that is provided on the side closer to the second plane P2 in the second pulley group 463a (the inner pulley of the second pulley group 463a) guide the first portion W1a1 of the first elongate element W1 engaged with the pulley portion 431a of the end effector member 430a. On the other hand, in FIG. 13, the pulley that is provided on the side far from the second plane P2 in the first pulley group 462b (the outer pulley of the first pulley group 462b) and the pulley that is provided on the side far from the second plane P2 in the second pulley group 463b (the outer pulley of the second pulley group 463b) guide the second portion W1a2 of the first elongate element W1 engaged with the pulley portion 431b of the end effector member 430b. The first pulley groups 462a and 462b are arranged between the second axis A2 and the first axis A1. The second pulley groups 463a and 463b are arranged on the second axis A2. More specifically, the cylindrical-column-shaped attachment W1b of the first elongate element W1 is engaged with the pulley portion 431a. When the first elongate element W1 moves, the pulley portion 431a rotates about the first rotational axis A1 and thus the end effector member 430a rotates about the first rotational axis A1. As illustrated in FIG. 12, the circular-column shaped attachment W2b of the second elongate element W2 is engaged with the pulley portion 431b. As the second elongate element W2 moves, the pulley portion 431b rotates about the first axis A1 and thus the end effector member 430b rotates about the first axis A1. The pulley that is provided on the side far from the second plane P2 in the first pulley group 462b (the outer pulley of the first pulley group 462b) and the pulley that is provided on the side farther from the second plane P2 in the second pulley group 463b (the outer pulley of the second pulley group 463b) guide the second portion W2a2 of the second elongate element W2 engaged with the pulley portion 431b of the end effector member 430b.

The first shaft portion 464a extends along a rotation axis (a rotation center) substantially parallel to the second shaft A2, and is arranged on the same side as the pulley portion 431a with respect to the first plane P1. The first shaft portion 464b is arranged on the same side as the pulley portion 431b with respect to the first plane P1. The second shaft portion 465 is a shaft member formed in a cylindrical column shape extending along the second axis A2. The second shaft portion 465 is inserted in and supported by a pair of shaft holes of the proximal clevis 470.

Here, each of the first elongate element W1, the second elongate element W2, and the third elongate element is a wire or a cable. Each of the first elongate element W1, the second elongate elements W2, and third elongate element W3 is made of a metal such as stainless steel, tungsten, or the like. The third elongate element W3 is provided corresponding to the pulley portion 461. The first elongate element W1 and the second elongate element W2 are provided corresponding to the end effector members 430a and 430b. Note that a part of each of the first elongate element W1, the second elongate elements W2, and third elongate element W3 may be made of a rod or the like.

As illustrated in FIG. 11, the proximal clevis 470 includes a connection base portion 470a connected to the shaft 420.

As illustrated in FIG. 14, the arm 60 is provided with the plurality of servomotors M1, a plurality of encoders E1, and a plurality of speed reducers (not illustrated), so as to correspond to the plurality of joint portions 64 of the arm section 61. The encoder E1 is configured to detect the rotation angle of the servomotor M1. The speed reducer is configured to reduce the rotation of the servomotor M1 to increase the torque.

As illustrated in FIG. 14, the translational movement mechanism 70 includes: four servomotors M2 (which are servomotors 712a1 to 712d1 illustrated in FIG. 9) for rotating the rotation members 44a to 44d provided on the surgical instrument 4; a servomotor M3 for translating the surgical instrument 4; four encoders E2 and an encoder E3; and speed reducers (the speed reducers 712a2 to 712d2 corresponding to the servomotors 712a1 to 712d1 illustrated in FIG. 9 and a speed reducer (not illustrated) corresponding to the servomotor M3). The four encoder E2 are configured to detect the rotation angles of the servomotors 712a1 to 712d1 illustrated in FIG. 9. The encoder E3 is configured to detect the rotation angle of the servomotor M3. The speed reducers are configured to reduce the rotation speeds of the servomotors M2 and the servomotor M3 to increase the torque thereof.

The positioner 40 is provided with a plurality of servomotors M4, a plurality of encoders E4, and a plurality of speed reducers (not illustrated), so as to correspond to the plurality of joints 43 of the positioner 40. The encoders E4 detect the rotation angles of the servomotors M4. The speed reducers are configured to reduce the rotations of the servomotors M4 to increase the torque thereof.

The medical trolley 3 is provided with servomotors M5 that drive a plurality of front wheels (not illustrated) of the medical trolley 3 respectively, encoders E5, and speed reducers (not illustrated). The encoders E5 detect the rotation angles of the servomotors M5. The speed reducer is configured to reduce the rotation of the servomotor M5 to increase the torque.

The control unit 31 of the medical trolley 3 includes an arm control unit 31a that controls the movement of the plurality of arms 60 based on commands, and a positioner control unit 31b that controls the movement of the positioner 40 and driving of the front wheel (not illustrated) of the medical trolley 3 based on commands. A servo control unit C1 that controls the servomotors M1 for driving the arm 60 is electrically connected to the arm control unit 31a. Further, an encoder E1 that detects the rotation angle of the servomotor M1 is electrically connected to the servo control unit C1.

A servo control unit C2 that controls the servomotors M2 for driving the surgical instrument 4 is electrically connected to the arm control unit 31a. The encoders E2 that detect the rotation angles of the servomotors M2 are electrically connected to the servo control unit C2. The servo control unit C3 that controls the servomotor M3 for translationally moving by the translational movement mechanism 70 is electrically connected to the arm control unit 31a. The encoder E3 for detecting the rotation angle of the servomotor M3 is electrically connected to the servo control unit C3.

The operation command input to the remote control apparatus 2 is input to the arm control unit 31a. The arm control unit 31a generates position commands based on the operation command inputted and the rotation angles detected by the encoders E1 (E2, E3), and outputs the position commands to the servo control units C1 (C2, C3). The servo control units C1 (C2, C3) generate torque commands based on the position commands inputted from the arm control unit 31a and the rotation angles detected by the encoders E1 (E2, E3), and output the torque commands to the servomotors M1 (M2, M3). As a result, the arm 60 is moved so as to comply with the operation command inputted to the remote control apparatus 2.

The arm control unit 31a is configured to operate the arm 60 based on an input signal from the operation unit 80. Specifically, the arm control unit 31a generates position commands based on the input signal (operation command) inputted from the operation unit 80 and the rotation angles detected by the encoders E1 or E3, and outputs the position commands to the servo control units C1 or C3. The servo control units C1 or C3 generate torque commands based on the position command inputted from the arm control unit 31a and the rotation angles detected by the encoders E1 or E3, and outputs the generated torque commands to the servomotors M1 or M3. As a result, the arm 60 is moved so as to follow the operation command inputted to the operation unit 80.

As illustrated in FIG. 14, the servo control units C4 that control the servomotors M4 for moving the positioner 40 are electrically connected to the positioner control unit 31b. The encoders E4 that detects the rotation angles of the servomotors M4 are electrically connected to the servo control units C4. The servo control units C5 that control the servomotors 5 for driving the front wheel (not illustrated) of the medical trolley 3 are electrically connected to the positioner control unit 31b. The encoders E5 that detect the rotation angles of the servomotors M5 are electrically connected to the servo control units C5.

An operation command is input from the input device 33 to the positioner control unit 31b. The positioner control unit 31b generates position commands based on the operation command inputted from the input device 33 and the rotation angle detected by the encoder E4, and outputs the position commands to the servo control units C4. The servo control unit C4 generates torque commands based on the position command input from the positioner control unit 31b and the rotation angles detected by the encoders E4, and outputs the torque commands to the servomotors M4. As a result, the positioner 40 is moved so as to follow the operation command input to the input device 33. Similarly, although detailed explanation is omitted, the positioner control unit 31b moves the medical trolley 3 based on the operation command inputted from the operation handle 34.

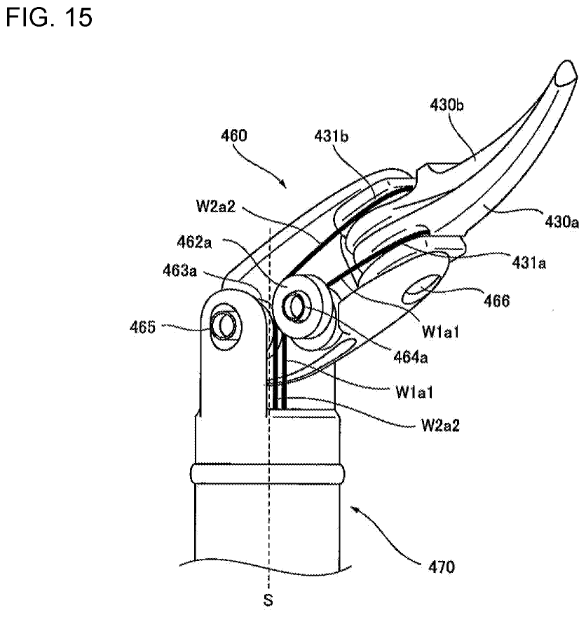

FIG. 15 is a diagram illustrating a view in which the distal clevis 460, which is the wrist of the surgical instrument 4, is rotated in the C6a direction (see FIG. 12) about the second shaft portion 465. The surgical instrument 4 is configured, when the rotation member 44c is rotated in the C2 direction (see FIG. 10), to pull the second portion W1a2 of the first elongate element W1 to drive the end effector member 430a to rotate in the direction of closing the end effector member 430a, and is configured, when the rotation member 44d is rotated in the C4 direction (see FIG. 10), to pull the second portion W2a2 of the second elongate element W2 to drive the end effector member 430b to rotate in the direction of closing the end effector member 430b. In a case where the surgical instrument 4 is scissors, a shearing force is generated between the end effector members 430a and 430b (the scissors) by further rotating the rotation member 44c in the C2 direction by a predetermined rotation angles (a first predetermined rotation angle) and the rotation member 44d in the C4 direction by a predetermined rotation angles (a second predetermined rotation angle) after the end effector members 430a and 430b are closed. In a case where the surgical instrument 4 is graspers, a gripping force is generated between the end effector members 430a and 430b (the graspers) by further rotating the rotation member 44c in the C2 direction by a predetermined rotation angles (a first predetermined rotation angle) and the rotation member 44d in the C4 direction by a predetermined rotation angles (a second predetermined rotation angle) after the end effector members 430a and 430b are closed. Note that the predetermined rotation angle and the second predetermined rotation angle may be the same.

As illustrated in FIG. 12, when the distal clevis 460 is not rotated about the second shaft portion 465, the second portion W2a2 of the second elongate element W2 for driving the end effector member 430b in the closing direction is guided by both the outer pulley of the first pulley group 462a and the outer pulley of the second pulley group 463a (hereinafter, may be referred to as a first state). On the other hand, as illustrated in FIG. 15, when the distal clevis 460 is rotated about the second shaft portion 465 by a predetermined angle in the C6a direction (see FIG. 12), the second portion W2a2 of the second elongate element W2 for driving the end effector member 430b in the closing direction is guided by the outer pulley of the first pulley group 462a (hereinafter, may be referred to as a second state). Each pulley included in the first pulley group 462a has a smaller diameter than each pulley included in the second pulley group 463a. Accordingly, the path length of the second portion W2a2 of the second elongate element W2 (the length of the second portion W2a2 between the end effector member 430b and the rotation member 44d) is shorter in the second state than in the first state. Therefore, in the first state, a shearing force is generated when the rotation member 44d is further rotated by the predetermined rotation angle (X degrees) in the C4 direction after the end effector members 430a and 430b are closed. However, in the second state, the path length of the second portion W2a2 becomes shorter than the first state and thus the second portion W2a2 loosens, so that the shearing force may become insufficient even if the rotation member 44d is rotated by the predetermined rotation angle (X degrees) in the C4 direction after the end effector members 430a and 430b are closed.

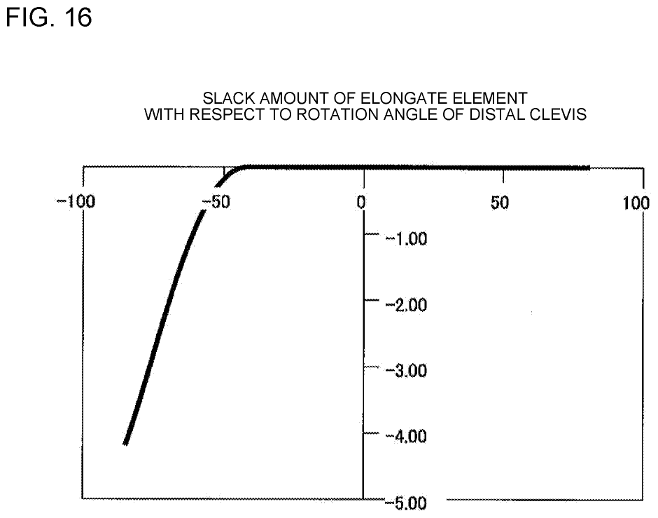

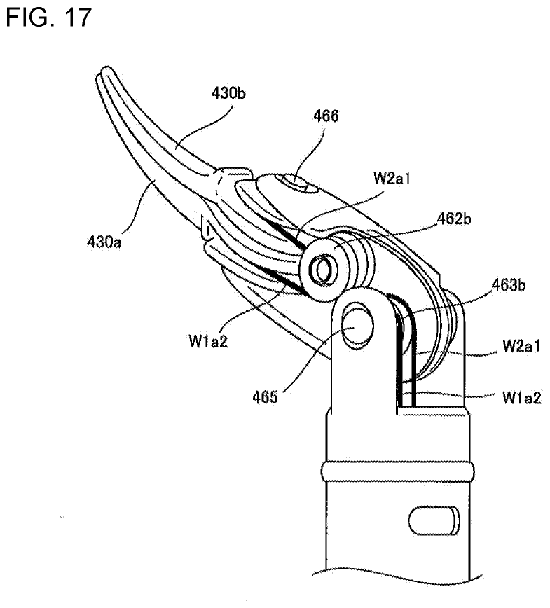

FIG. 16 is a diagram illustrating a relationship between the rotation angle (bending angle) of the distal clevis 460 about the second shaft portion 465 and a loosening amount (an amount of slack) of the second portion W2a2 of the second elongate element W2. In FIG. 16, the horizontal axis indicates the rotation angle of the distal clevis 460 about the second shaft portion 465, and the vertical axis indicates the rotation angle of the rotation member 44d corresponding to and converted from the loosening amount (the slack amount) of the second elongate element W2. It can be seen that when the distal clevis 460 is rotated by about 50 degrees in the C6a direction (see FIG. 12) from the state illustrated in FIG. 16, the second portion W2a2 becomes loose, and as the rotation angle of the distal clevis 460 further increases, the loosening amount of the second portion W2a2 gradually increases. This is because, as the distal clevis 460 rotates in the C6a direction, the guide amount of the second portion W2a2 guided by the outer pulley (having the small diameter) of the first pulley group 462a increases while the guide amount of the second portion W2a2 guided by the outer pulley (having the large diameter) of the second pulley group 463a increases. Therefore, in the case where the surgical instrument is scissors, in order to exert the predetermined shearing force, it may be preferable to correct the loosening amount of the second portion W2a2 according to the rotation angle of the distal clevis 460 in the C6a direction. Specifically, the control unit 31 controls the drive of the actuator 712d corresponding to the rotation member 44d so as to increase the rotation amount (the rotation angle) of the rotation member 44d in the C4 direction by the amount corresponding to the loosening amount of the second portion W2a2 due to the rotation angle of the distal clevis 460. Note that when the distal clevis 460 rotates in the C5a direction, the second portion W1a2 of the first elongate element W1 becomes loose. Accordingly, the control unit 31 controls the drive of the actuator 712c for rotating the rotation member 44c so that the amount of rotation (rotation angle) of the rotation member 44c in the C2 direction increases by the amount corresponding to the loosening amount of the second portion W1a2 of the first elongate element W1 caused by the rotation angle of the distal clevis 460. FIG. 17 is a view of the end effector 430 illustrated in FIG. 15 as viewed from the opposite side with respect to the second plane P2 illustrated in FIG. 11. As illustrated in FIG. 17, when the distal clevis 460 of the surgical instrument 4 is rotated about the second shaft portion 465 by a predetermined angle in the C6a direction (see FIG. 12), the second portion W1a2 of the first elongate element W1 is guided by both the outer pulley of the first pulley group 462b and the outer pulley of the second pulley group 463b. Therefore, the path length of the second portion W1a2 of the first elongate element W1 driven to close the end effector member 430a does not change, and thus it is not necessary to correct the rotation amount of the rotation member 44c in the C2 direction.

When the distal clevis 460 of the surgical instrument 4 is rotated about the second shaft portion 465 in the C5a direction (see FIG. 12), the control unit 31 controls the drive of the actuator 712c for rotating the rotation member 44c so as to increase the rotation amount (the rotation angle) of the rotation member 44c in the C2 direction by the amount corresponding to the loosening amount caused by the rotation angle of the distal clevis 460. In this case, since the path length of the second portion W2a2 of the second elongate element W2 driven to close the end effector member 430b does not change, it is not necessary to correct the rotation amount of the rotation member 44d in the C4 direction.

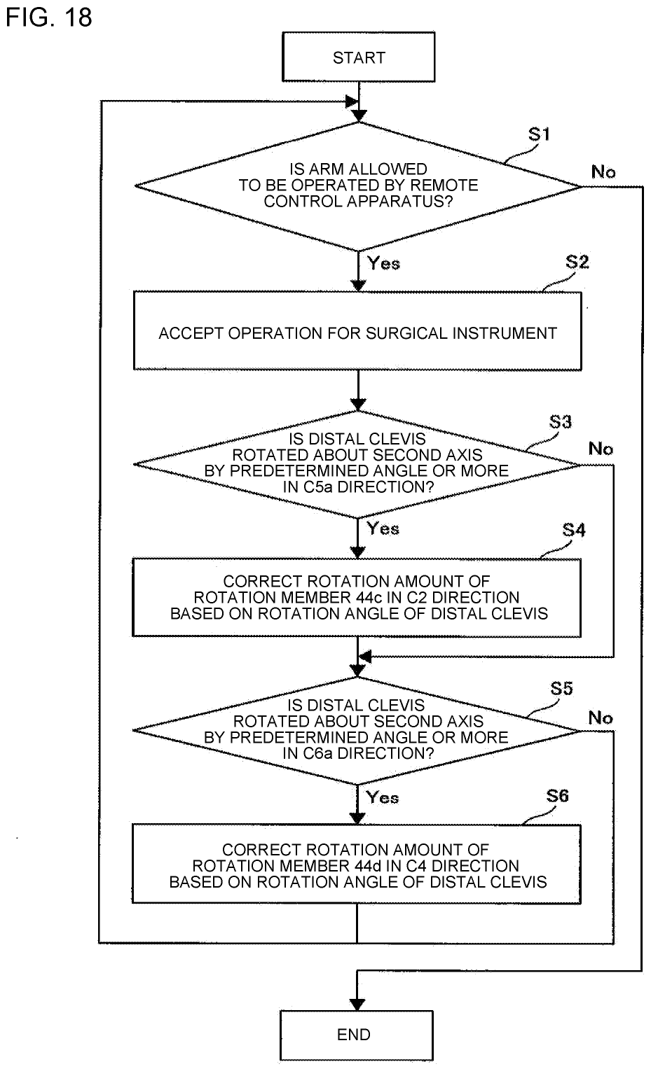

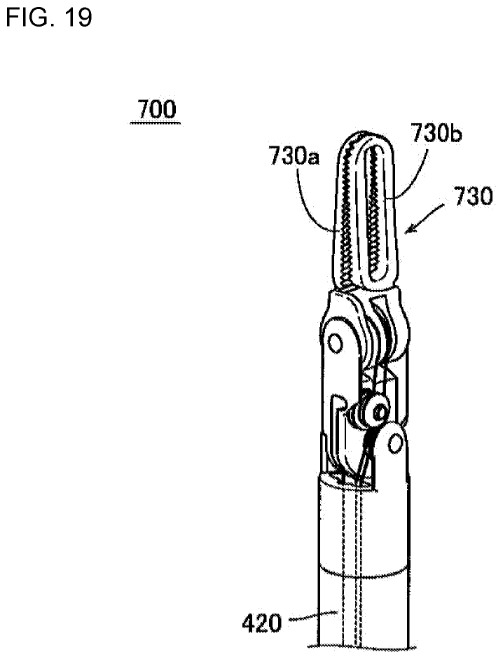

Next, with reference to FIG. 18, a control method of the surgical system 100 is described below.

First, in step S1, the control unit 31 determines whether or not the arm 60 of the medical manipulator 1 is allowed to be operated by the remote control apparatus 2. For example, when a sensor(s) provided in the vicinity of the monitor 24 detects the head of the operator and the operator operates the operation manipulator arm(s) 21 of the remote control apparatus 2, the control unit 31 determines that the arms 60 are allowed to be operated by the remote control apparatus 2.

Next, in step S2, the control unit 31 accepts an operation (e.g., a user input) of the operation manipulator arm(s) 21 of the remote control apparatus 2 to operate the surgical instrument 4.

Next, in step S3, the control unit 31 determines, based on the accepted operation, whether or not to rotate the distal clevis 460 about the second shaft portion 465 (the second axis A2) by the predetermined angle or more in the C5a direction. Specifically, the control unit 31 monitors (detects) the rotation direction and rotation angle of the servomotor 712b1 of the actuator 712b that rotates the rotation member 44b by the encoder E2, and determines, based on the detection result, whether or not the distal clevis 460 rotates about the second shaft portion 465 in the C5a direction by the predetermined angle or more. When it is determined that the distal clevis 460 rotates in the C5a direction by the predetermined angle or more, the process proceeds to step S4 and when it is not determined that the distal clevis 460 rotates in the C5a direction by the predetermined angle or more, the process proceeds to step S5.

Next, in step S4, the control unit 31 controls the drive of the actuator 712c for driving the rotation member 44c so as to increase the rotation amount (the rotation angle) of the rotation member 44c in the C2 direction by the amount corresponding to the loosening amount of the second portion W1a2 of the first elongate element W1. Note that, as described above, the loosening amount of the second portion W1a2 of the first elongated element W1 corresponds to the rotation angle of the distal clevis 460 about the second shaft portion 465 by the predetermined angle or more in the C5a direction.

Next, in step S5, the control unit 31 determines, based on the accepted operation, whether or not to rotate the distal clevis 460 about the second shaft portion 465 (the second axis A2) by the predetermined angle or more in the C6a direction. When it is determined that the distal clevis 460 rotates in the C6a direction by the predetermined angle or more, the process proceeds to step S6 and it is not determined that the distal clevis 460 rotates in the C5a direction by the predetermined angle or more, the process proceeds to step S1.

In step S6, the control unit 31 controls the drive of the actuator 712d corresponding to the rotation member 44d so as to increase the rotation amount (the rotation angle) of the rotation member 44d in the C4 direction by the amount corresponding to the loosening amount of the second portion W2a2 of the second elongate element W2. Note that, the loosening amount of the second portion W2a2 of the second elongated element W2 corresponds to the rotation angle of the distal clevis 60 about the second shaft portion 465 by the predetermined angle or more in the C6a direction.

Steps S2 to S6 described above are performed for each of the arms 60 corresponding to the two operation manipulator arms 21.

### Effects of Embodiments

According to one or more embodiments, effects as described below can be obtained.

In one or more embodiments described above, the control unit 31 determines whether or not the distal clevis 460 rotates about the second shaft portion (the second axis A2) by the predetermined angle or more, and when it is determined that the distal clevis 460 rotates by the predetermined angle or more, controls the corresponding actuator 712c or 712d so as to increase the rotation angle of the rotation member 44c or 44d. Therefore, it is possible to suppress a decrease in the shearing force of the scissors, a decrease in the gripping force of the graspers, or the like due to the slack of the elongate element W.

[Modifications]

Note that one or more embodiments disclosed herein should be considered as exemplary in all respects and do not limit the invention. The scope of the invention is indicated by claims, not by explanation of one or more embodiments described above, and includes equivalents to the claims and all alterations (modification) within the same.

For example, the control unit 31 may monitor the rotation angle of the shaft 420 and control the rotation angles of the rotation member 44c and the rotation member 44d according to the rotation angle of the shaft 420. Specifically, the path length of the second portion W1a2 of the first elongate element W1 and the path length of the second portion W2a2 of the second elongate element W2 become longer in the state where the shaft 420 is rotated about the axis S of the shaft 420 from the initial position than in the state where the shaft 420 is in the initial position. This is because the first elongate elements W1 and W2 do not pass through the center of the shaft 420 when the shaft 420 is rotated. Therefore, the control unit 31 controls the rotation amount (the rotation angle) of the rotation member 44c and the rotation member 44d to be decreased according to the rotation angle of the shaft 20 from the initial position thereof.

Further, in an embodiment described above, the case has been described in which the surgical instrument 4 is configured to drive the rotation member 44c to rotate in the C2 direction (see FIG. 10) to pull the second portion W1a2 of the first elongate element W1 so as to rotate the end effector member 430a in the closing direction thereof and to drive the rotation member 44d to rotate in the C4 direction (see FIG. 10) to pull the second portion W2a2 of the second elongate element W2 so as to rotate the end effector member 430b in the closing direction thereof. However, the disclosure is not limited thereto. For example, the surgical instrument may be configured having the paths of the first elongate elements W1 and W2 different from those in an embodiment described above such that the rotation member 44c is driven to rotate in the C1 direction (see FIG. 10) to rotate the end effector member 430a in the closing direction thereof and the rotation member 44d is driven to rotate in the C3 direction (see FIG. 10) to rotate the end effector member 430b in the closing direction thereof.

Further, in one or more embodiments described above, the case has been described in which the number of the arms 60 provided is four. However, the disclosure is not limited thereto. In the disclosure, the number of the arms 60 may be any number as long as at least one arm 60 is provided.

Further, in one or more embodiments described above, the case has been described in which each of the arm section 61 and the positioner 40 are configured as the 7-axis articulated robot. However, the disclosure is not limited thereto. For example, the arm section 61 and/or the positioner 40 may be configured as an articulated robot other than the 7-axis articulated robot (for example, a 6-axis articulated robot, an 8-axis articulated robot, or the like).

Further, in one or more embodiments described above, the case has been described in which the medical manipulator 1 includes the medical trolley 3, the positioner 40, the arm base 50, and the arms 60. However, the disclosure is not limited thereto. For example, the medical manipulator 1 may include only the arms 60 and not necessarily include the medical trolley 3, the positioner 40, and the arm base 50.

The functions of each of the elements disclosed herein may be carried out by a circuitry or a processing circuitry including a general purpose processor, a dedicated processor, an integrated circuit, an ASIC (Application Special Integrated Circuit), a conventional circuit, or a combination of two or more of them, that is configured or programmed to perform the functions. A processor is considered a processing circuitry or a circuitry because it contains transistors and other circuit elements. In the disclosure, a circuit, a unit, or a means may be either a hardware that is configured to perform the recited function(s) or a hardware that is programmed to perform the recited function(s). The hardware may be the hardware disclosed herein, or may be other known hardware that is programmed or configured to perform the function(s) described. If the hardware is a processor which is considered as a type of a circuit, a circuit, a means, or a unit is a combination of hardware and software, and the software is used to configure the hardware and/or the processor.

The invention includes other embodiments or modifications in addition to one or more embodiments and modifications described above without departing from the spirit of the invention. The one or more embodiments and modifications described herein are to be considered in all respects as illustrative, and not restrictive. The scope of the invention is indicated by the appended claims rather than by the foregoing description. Hence, all configurations including the meaning and range within equivalent arrangements of the claims are intended to be embraced in the invention.

1. A surgical system comprising:
a patient-side apparatus that includes a robot arm including, on a distal end side of the robot arm, an attachment portion to which a surgical instrument is attached;
an operator-side apparatus including an operation part configured to receive an operation for operating the surgical instrument; and
a controller configured to control an operation of the surgical instrument based on a command from the operation part, wherein
the surgical instrument includes:
a shaft;
a first support body connected to a distal end portion of the shaft;
a second support body supported at a distal end portion of the first support body and configured to be rotatable about a first axis orthogonal to an axial direction of the shaft with respect to the first support body;
a first jaw member including a first pulley portion supported at a distal end portion of the second support body and configured to be rotatable about a second axis orthogonal to the axial direction of the shaft and the first axis with respect to the second support body;
a second jaw member including a second pulley portion supported at the distal end portion of the second support body and configured to be rotatable about the second axis with respect to the second support body;
a first elongate element for operating the first jaw member;
a second elongate element for operating the second jaw member;
a first rotation member provided on a proximal end side of the shaft and configured to be rotated to drive the first elongate element; and
a second rotation member provided on the proximal end side of the shaft and configured to be rotated to drive the second elongate element,
the first jaw member and the second jaw member are configured to be closed to each other by the first pulley portion being rotated in a first direction and the second pulley portion being rotated in a second direction opposite to the first direction,
the attachment portion of the robot arm includes a first actuator configured to drive the first rotation member and a second actuator configured to drive the second rotation member of the surgical instrument attached to the attachment portion, and
upon receiving from the operation part a command to rotate the first and second rotation members by first predetermined rotation angle and second predetermined rotation angle respectively for closing the first and second jaw members to each other, the controller is configured, when a path length of the first elongate element between the first jaw member and the first rotation member changes, to control the first actuator to rotate the first rotation member by a first rotation angle corrected according to the change in the path length of the first elongate element, and when a path length of the second elongate element between the second jaw member and the second rotation member changes, to control the second actuator to rotate the second rotation member by a second rotation angle corrected according to the change in the path length of the second elongate element.

2. The surgical system according to claim 1, wherein
the surgical instrument includes a first pulley rotatable about the first axis and configured to guide the first elongate element, and a second pulley located between the distal end portion and a proximal end portion of the second support body and configured to guide the first elongate element, and
the first pulley and the second pulley are arranged so as to guide the first elongate element between the shaft and the first pulley portion of the first jaw member.

3. The surgical system according to claim 2, wherein
the first pulley portion is disposed on a first side with respect to a first plane including the first axis and being orthogonal to the second axis, the second pulley portion is disposed on a second side being opposite to the first side with respect to the first plane, and
a rotation axis of the second pulley is disposed on the first side with respect to the first plane.

4. The surgical system according to claim 3, wherein
the first pulley and the second pulley are disposed on a third side with respect to a second plane, the second plane including the second axis and being orthogonal to the first axis,
the surgical instrument includes:
a third pulley provided on a fourth side opposite to the third side with respect to the second plane and rotatably provided about the first axis; and
a fourth pulley provided between the distal end portion and the proximal end portion of the second support body, provided on the fourth side with respect to the second plane, and provided on the second side with respect to the first plane, and
the first elongate element includes: a first portion guided by the first pulley and the second pulley; a second portion guided by the third pulley and the fourth pulley; and a first attachment provided between the first portion and the second portion fixed to the first pulley portion of the first jaw member.

5. The surgical system according to claim 4, wherein
the second elongate element includes: a third portion; a fourth portion; and a second attachment provided between the third portion and the fourth portion and fixed to the second pulley portion of the second jaw member,
the surgical instrument includes:
a fifth pulley provided on the fourth side with respect to the second plane, configured to be rotatable about the first axis to guide the third portion of the second elongate element;
a sixth pulley provided between the distal end portion and the proximal end portion of the second support body, provided on the fourth side with respect to the second plane, and configured to guide the third portion of the second elongate element;
a seventh pulley provided on the third side with respect to the second plane and configured to be rotatable about the first axis to guide the fourth portion of the second elongate element; and
an eighth pulley provided between the distal end portion and the proximal end portion of the second support body, provided on the third side with respect to the second plane, and configured to guide the fourth portion of the second elongate element.

6. The surgical system according to claim 1, wherein
the surgical instrument includes:
a third elongate element that includes: a fifth portion; a sixth portion; and a third attachment provided between the fifth portion and the sixth portion and fixed to the second support body, and is configured to drive the second support body; and
a third rotation member provided on the proximal end side of the shaft and configured to be rotated to drive the third elongate element, and
the attachment portion of the robot arm includes a third actuator configured to drive the third rotation member of the surgical instrument attached to the attachment portion.

7. The surgical system according to claim 1, wherein
the first actuator includes a first motor and a first speed reducer configured to decelerate a rotation of the first motor at 1/10 to 1/150 times and output the decelerated rotation from the first speed reducer, and the second actuator includes a second motor and a second speed reducer configured to decelerate a rotation of the second motor at 1/10 to 1/150 times and output the decelerated rotation from the second speed reducer.

8. The surgical system according to claim 1, wherein
the controller is configured, when the path length of the first elongate element does not change, to control the first actuator to rotate the first rotation member by the first predetermined rotation angle, and when the path length of the second elongate element does not change, to control the second actuator to rotate the second rotation member by the second predetermined rotation angle.

9. The surgical system according to claim 1, wherein
the controller is configured, when the path length of the first elongate element becomes shorter than a first predetermined path length, to control the first actuator to rotate the first rotation member by the first rotation angle greater than the first predetermined rotation angle, and when the path length of the second elongate element becomes shorter than a second predetermined path length, to control the second actuator to rotate the second rotation member by the second rotation angle greater than the second predetermined rotation angle.

10. The surgical system according to claim 3, wherein
the controller is configured, when the second support body is rotated about the first axis toward the second side by a first angle or more, to control the first actuator to rotate the first rotation member by the first rotation angle greater than the first predetermined rotation angle according to the rotation angle of the second support body.

11. The surgical system according to claim 10, wherein
the surgical instrument is configured such that when the second support body is rotated by the first angle or more, the first elongated element is guided more by the second pulley than by the first pulley.

12. The surgical system according to claim 5, wherein
the controller is configured, when the second support body is rotated about the first axis toward the first side by a second angle or more, to control the second actuator to rotate the second rotation member by the second rotation angle greater than the second predetermined rotation angle.

13. The surgical system according to claim 3, wherein
the controller is configured, when the path length of the first elongate element becomes longer than a first predetermined path length, to control the first actuator to rotate the first rotation member by the first rotation angle smaller than the first predetermined rotation angle, and when the path length of the second elongate element becomes longer than a second predetermined path length, to control the second actuator to rotate the second rotation member by the second rotation angle smaller than the second predetermined rotation angle.

14. The surgical system according to claim 1, wherein
the shaft is provided to be rotatable about an axis of the shaft,
the controller is configured, when the shaft has not been rotated from an initial position of the shaft, to control the first actuator and the second actuator to rotate the first rotation member and the second rotation member by the first predetermined rotation angle and the second predetermined rotation angle respectively and, when the shaft has been rotated from the initial position of the shaft, to control the first actuator and the second actuator to rotate the first rotation member and the second rotation member by the first rotation angle smaller than the first predetermined rotation angle and the second rotation angle smaller than the second predetermined rotation angle respectively according to the rotation angle of the shaft.

15. A surgical system comprising:
a patient-side apparatus that includes a robot arm including, on a distal end side of the robot arm, an attachment portion to which a surgical instrument is attached;
an operator-side apparatus including an operation part configured to receive an operation for operating the surgical instrument;
a controller configured to control an operation of the surgical instrument based on a command from the operation part, wherein
the surgical instrument includes:
a shaft;
a first support body connected to a distal end portion of the shaft;
a second support body supported at a distal end portion of the first support body and configured to be rotatable about a first axis orthogonal to an axial direction of the shaft with respect to the first support body;
a first jaw member including a first pulley portion supported at a distal end portion of the second support body and configured to be rotatable about a second axis orthogonal to the axial direction of the shaft and the first axis with respect to the second support body;
a second jaw member including a second pulley portion supported at the distal end portion of the second support body and configured to be rotatable about the second axis with respect to the second support body;
a first elongate element for operating the first jaw member;
a second elongate element for operating the second jaw member;
a first rotation member provided on a proximal end side of the shaft and configured to be rotated to drive the first elongate element; and
a second rotation member provided on the proximal end side of the shaft and configured to be rotated to drive the second elongate element; and
the first jaw member and the second jaw member are configured to be closed to each other by the first pulley portion being rotated in a first direction and the second pulley portion being rotated in a second direction opposite to the first direction,
the first pulley portion is disposed on a first side with respect to a first plane including the first axis and being orthogonal to the second axis, the second pulley portion is disposed on a second side being opposite to the first side with respect to the first plane, and
the attachment portion of the robot arm includes a first actuator configured to drive the first rotation member and a second actuator configured to drive the second rotation member of the surgical instrument attached to the attachment portion, and
upon receiving from the operation part a command to rotate the first and second rotation members by first predetermined rotation angle and second predetermined rotation angle respectively for closing the first and second jaw members to each other, the controller is configured, when the second support body is rotated about the first axis toward the second side by a first angle or more, to control the first actuator to rotate the first rotation member by a first rotation angle greater than the first predetermined rotation angle according to the rotation angle of the second support body and to control the second actuator to rotate the second rotation member by the second predetermined rotation angle.

16. The surgical system according to claim 15, wherein
the controller is configured, when the second support body is rotated about the first axis toward the first side by the first angle or more, to control the first actuator to rotate the first rotation member by the first predetermined rotation angle and to control the second actuator to rotate the second rotation member by a second rotation angle greater than the second predetermined rotation angle according to the rotation angle of the second support body.

17. The surgical system according to claim 15, wherein
the surgical instrument include a first pulley rotatable about the first axis and configured to guide the first elongate element, and a second pulley located between the distal end portion and a proximal end portion of the second support body and configured to guide the first elongate element.

18. The surgical system according to claim 17, wherein
a rotation axis of the second pulley is disposed on the first side with respect to the first plane,
the first pulley and the second pulley are disposed on a third side with respect to a second plane, the second plane including the second axis and orthogonal to the first axis,
the surgical instrument includes: a third pulley provided on a fourth side opposite to the third side with respect to the second plane, rotatably provided about the first axis to guide the first elongate element; and
a fourth pulley provided between the distal end portion and the proximal end portion of the second support body, provided on the fourth side with respect to the second plane, provided on the second side with respect to the first plane, and configured to guide the first elongate element.

19. A surgical system comprising:
a patient-side apparatus that includes a robot arm including, at a distal end side of the robot arm, an attachment portion to which a surgical instrument is attached;
an operator-side apparatus including an operation part configured to receive an operation for operating the surgical instrument;
a controller configured to control an operation of the surgical instrument based on a command from the operation part, wherein
the surgical instrument includes:
a shaft provided to be rotatable about an axis of the shaft;
a first support body connected to a distal end portion of the shaft;
a second support body supported at a distal end portion of the first support body and configured to be rotatable about a first axis orthogonal to an axial direction of the shaft with respect to the first support body;
a first jaw member including a first pulley portion supported at a distal end portion of the second support body and configured to be rotatable about a second axis orthogonal to the axial direction of the shaft and the first axis with respect to the second support body;
a second jaw member including a second pulley portion supported at the distal end portion of the second support body and configured to be rotatable about the second axis with respect to the second support body;
a first elongate element for operating the first jaw member;
a second elongate element for operating the second jaw member;
a first rotation member provided on a proximal end side of the shaft and configured to be rotated to drive the first elongate element; and
a second rotation member provided on the proximal end side of the shaft and configured to be rotated to drive the second elongate element; and
the first jaw member and the second jaw member are configured to be closed to each other by the first pulley portion being rotated in a first direction and the second pulley portion being rotated in a second direction opposite to the first direction,
the attachment portion of the robot arm includes a first actuator configured to drive the first rotation member and a second actuator configured to drive the second rotation member of the surgical instrument attached to the attachment portion, and
upon receiving a command to rotate the first and second rotation members by first and second predetermined rotation angles respectively for closing the first and second jaw members to each other, the controller is configured, when the shaft has been rotated about the axis of the shaft from an initial position of the shaft, to control the first actuator and the second actuator to rotate the first rotation member and the second rotation member by a first rotation angle smaller than the first predetermined rotation angle and a second rotation angle smaller than the second predetermined rotation angle respectively according to the rotation angle of the shaft.

20. The surgical system according to claim 19, wherein
the controller is configured, when the shaft has not been rotated from the initial position of the shaft, to control the first actuator and the second actuator to rotate the first rotation member and the second rotation member by the first predetermined rotation angle and the second predetermined rotation angle respectively.
